(19) United States
(12) Patent Application Publication
(10) Pub. No.: US 2004/0073148 A1
(43) Pub. Date: Apr. 15, 2004

(54) Leg sling

(76) Inventors: Wayne M. Urban, Bedford, OH (US); Robert M. Webb, Cleveland, OH (US)

Correspondence Address: CALFEE HALTER & GRISWOLD, LLP, 800 SUPERIOR AVENUE, SUITE 1400, CLEVELAND, OH, 44114, US

(21) Appl. No.: 10/269,300

(22) Filed: Oct. 11, 2002

(51) Int. Cl. A61F 5/00

(52) U.S. Cl. 602/4

(57) ABSTRACT

A leg sling (10) for supporting a patient's leg includes a leg portion (16) connectable with the patient's leg and a shoulder portion (12) extensible across a shoulder of the patient to transfer weight from the leg sling to the patient's shoulder. An elastic intermediate portion (14) is connected between the leg portion (16) and the shoulder portion (12) to transfer weight from the leg portion to the shoulder portion. In one embodiment, a first connector (70) releasably connects the intermediate portion (14) of the leg sling (10) with the shoulder portion (12) of the leg sling. A second connector (90) releasably connects the intermediate portion (14) of the leg sling (10) with the leg portion (16) of the leg sling.

### BACKGROUND OF THE INVENTION

1. Technical Field

The present invention relates to a medical restraining device. In particular, the present invention relates to a sling for supporting the leg of a user off the ground to prevent weight from being applied to the leg, for example, after a fracture of a leg bone.

2. Description of the Prior Art

Hallstedt U.S. Pat. No. 2,543,847 describes an appliance indicated to be “for use in the treatment of unilateral leg diseases which will eliminate all weight-bearing by the afflicted leg and at the same time permit reasonably great freedom of movement by the patient.”

Tiede et al. U.S. Pat. No. 5,172,703 describes a soft-tissue corrective aide supported at the hips and shoulders and including a plurality of resilient straps which mount to shoes or other anchoring appliances containing mating fasteners.

### SUMMARY OF THE INVENTION

The present invention relates to a leg sling for supporting a patient's leg. In one embodiment, the leg sling includes a leg portion connectable with the patient's leg to transfer load from the patient's leg to the leg sling. A shoulder portion of the leg sling is extensible across a shoulder of the patient to transfer load from the leg sling to the patient's shoulder. An elastic intermediate portion is connected between the leg portion and the shoulder portion to transfer load from the leg portion to the shoulder portion. The leg sling is preferably free of portions extensible around the patient's waist when in use by the patient.

In another embodiment, a leg sling in accordance with the invention includes a leg portion connectable with the patient's leg for movement with the patient's leg, an elastic intermediate portion, and a shoulder portion extensible across a shoulder of the patient for transferring load from the leg sling to the shoulder of the patient. In this embodiment, a connector preferably releasably connects at least one of the leg portion to the intermediate portion or the intermediate portion to the shoulder portion. More preferably, a first connector releasably connects the intermediate portion of the leg sling with the shoulder portion of the leg sling and a second connector releasably connects the intermediate portion of the leg sling with the leg portion of the leg sling.

### BRIEF DESCRIPTION OF THE DRAWINGS

The foregoing and other features of the present invention will become apparent to one skilled in the art to which the present invention relates upon consideration of the following description of the invention with reference to the accompanying drawings, in which:

FIG. 1 is a schematic illustration of a leg sling in accordance with the present invention, shown in use on a patient;

FIG. 2 is an enlarged view of a portion of FIG. 1, also showing the patient's leg and the leg sling in a second position;

FIG. 3 is an exploded perspective view of the leg sling of FIG. 1; and

FIG. 4 is a partially assembled view of the leg sling of FIG. 1.

### DETAILED DESCRIPTION OF THE INVENTION

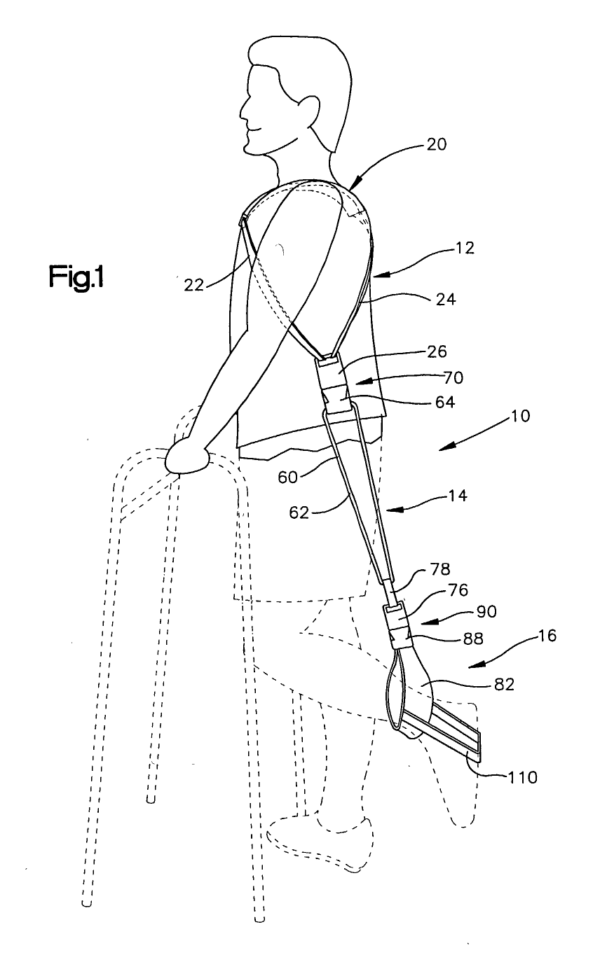

The present invention relates to a leg sling. In particular, the present invention relates to a sling for supporting the leg of a user off the ground so that the user's leg is maintained off of the ground with virtually no effort by the user. This is particularly important during recuperation after certain leg injuries, for example, after a fracture of a leg bone. As representative of the present invention, FIG. 1 illustrates a leg sling 10 constructed in accordance with the invention. The leg sling 10 preferably includes a shoulder portion or shoulder assembly 12, an intermediate portion 14, and a leg portion or leg assembly 16.

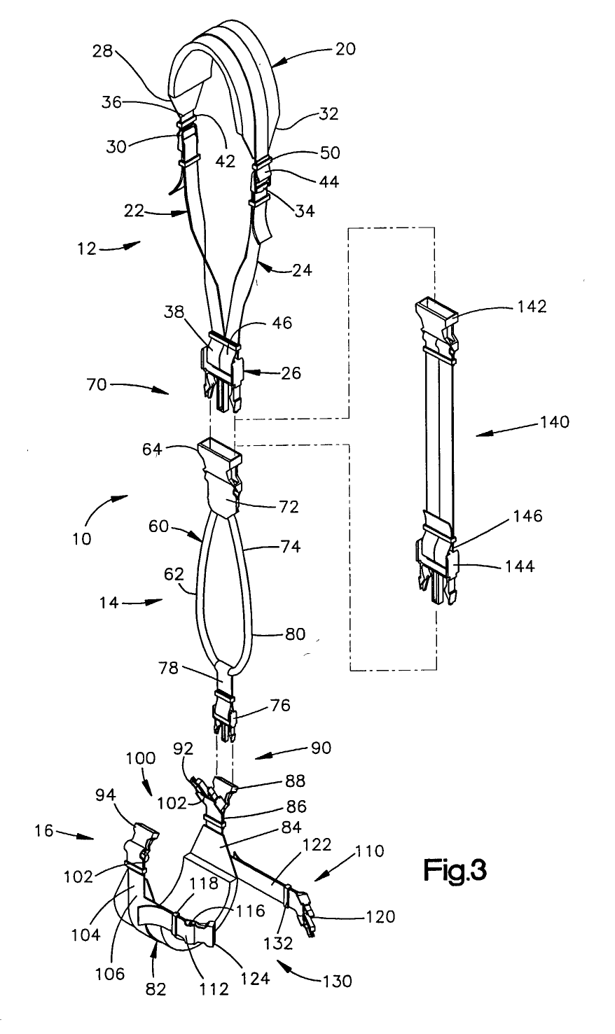

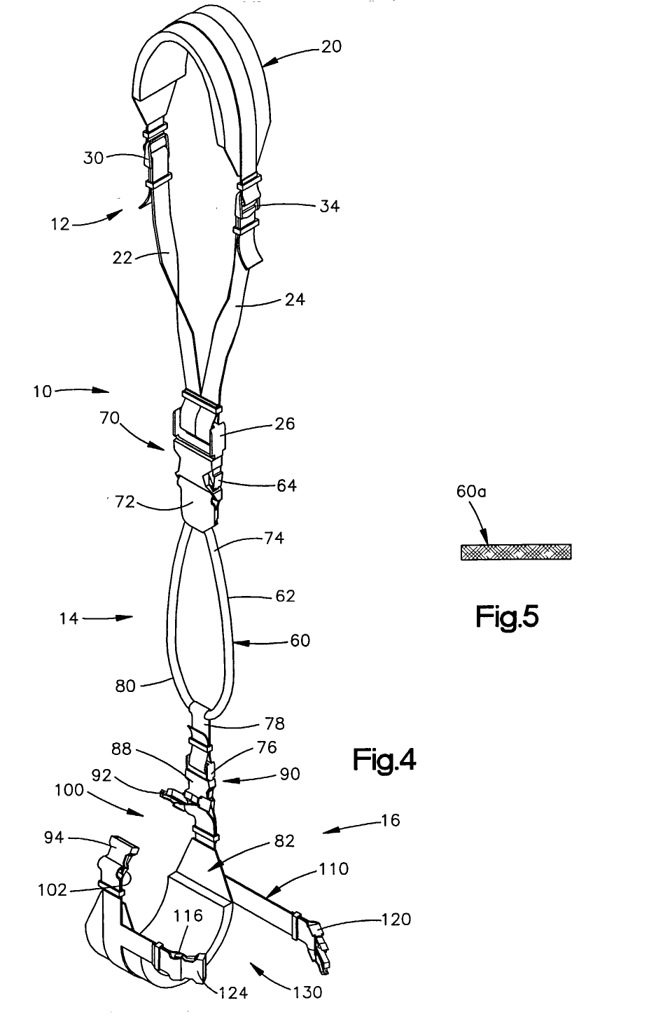

The shoulder assembly 12 of the leg sling 10 is adapted to fit over a shoulder of a patient. The shoulder assembly 12 (FIGS. 3 and 4) preferably includes a shoulder pad 20, a front strap 22, a back strap 24, and a first tongue assembly 26.

The shoulder pad 20 is preferably relatively wide, for example two to three inches, and thus is adapted to spread load from the leg sling 10 over an area larger than that of a corresponding strap and preferably over a relatively large area of the patient's shoulder. The shoulder pad 20 is preferably fixed in length. A first end portion 28 of the shoulder pad 20 is preferably fixed to a front strap adjuster 30. A second end portion 32 of the shoulder pad 20 is preferably fixed to a back strap adjuster 34.

The front strap 22, in the illustrated embodiment, is preferably made from webbing about one inch wide. A first end portion 36 of the front strap 22 preferably extends through the front strap adjuster 30 and is preferably selectively positionable relative to the front strap adjuster. A second end portion 38 of the front strap 22 is preferably looped around and thereby fixed to the first tongue assembly 26.

The front strap 22 is preferably adjustable in length by moving the first end portion 36 of the front strap through the front strap adjuster 30. The first end portion 36 of the front strap 22 is preferably secured by a tie down 42 on the front strap. When this is done, the length of the front strap 22 is set.

The back strap 24 of the shoulder assembly 12 is preferably similar in construction to the front strap 22. A first end portion 44 of the back strap 24 extends through the back strap adjuster 34 and is preferably selectively positionable relative to the back strap adjuster. A second end portion 46 of the back strap 24 is preferably looped around and thereby fixed to the first tongue assembly 26.

The back strap 24 is preferably adjustable in length by moving the first end portion 44 of the back strap through the back strap adjuster 34. The first end portion 44 of the back strap 24 is preferably secured by a tie down 50 on the back strap. When this is done, the length of the back strap 24 is set.

The shoulder pad 20 is thus disposed intermediate the front strap 22 and the back strap 24. Because the length of the front and back straps 22 and 24 can be adjusted, the shoulder pad 20 can be positioned anywhere along the length of the shoulder assembly 12, relative to the first tongue assembly 26, for maximum effect and comfort.

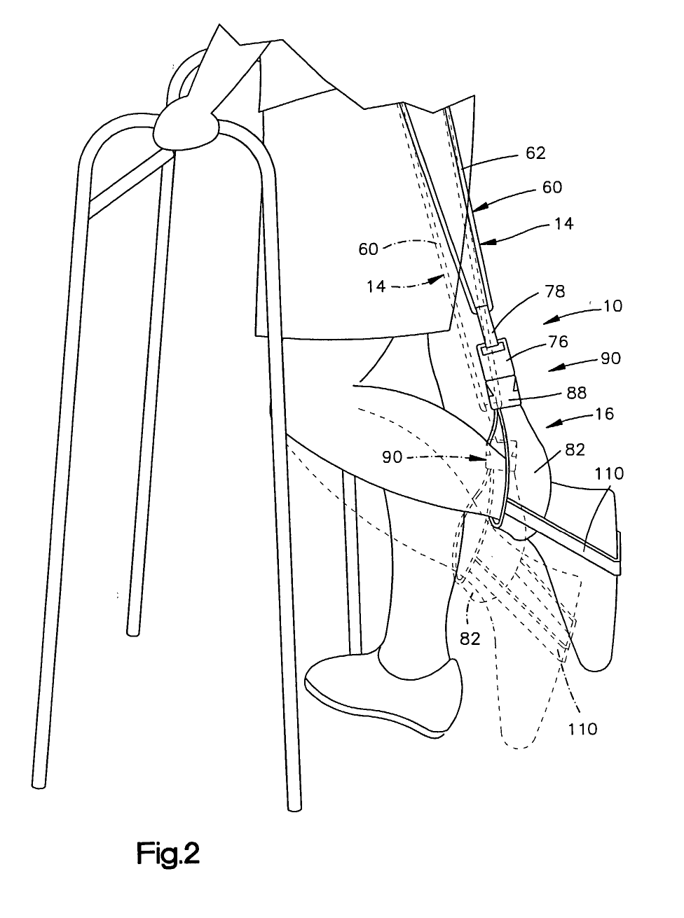

The intermediate portion 14 of the leg sling 10 preferably includes an elastic strap or elastic member 60. The elastic member 60, in the illustrated embodiment, is made from an elastic material, such as elastic shock cord, commonly referred to as “bungee cord.” This material is commonly available in various lengths with a generally circular cross-sectional configuration, as illustrated. This material elongates under load then recoils to its original length. The amount of elongation of the elastic member 60 under a given load is selected to hold the leg at the desired distance off the ground temporarily while walking using the unsupported leg, while preferably also allowing the supported leg to be placed on the ground by extending the leg with normal muscular action, e.g., by flexing the quadriceps, to overcome the leg-lifting force exerted by the elastic member 60. The portions of FIG. 2 in dot-dash lines show a leg and sling 10 moving toward the standing position against the force exerted by the elastic member 60.

The elastic member 60 in the illustrated embodiment is formed as a loop 62 of the shock cord. In the illustrated embodiment, the loop 62 preferably has a length when pulled taut but not stretched of about ten inches. The loop 62 may elongate by up to about 100% or more under load applied by the patient's leg muscles.

It should be understood that the intermediate portion 14 of the leg sling 10, including the elastic member 60, could have a different construction and/or configuration than that illustrated. For example, a different material than shock cord could be used for the elastic member 60, such as a flat or strap-like material, or a rubber material, rather than a round fabric material. In addition, the elastic member 60 could be a single length of material extending between the shoulder assembly 12 and the leg assembly 16, rather than a loop of material as illustrated. FIG. 5 illustrates, for example, a cross-section of a flat shock cord 60a.

The intermediate portion 14 of the leg sling 10 preferably includes a first buckle 64 that is adapted to receive the first tongue assembly 26 on the shoulder portion 12 of the leg sling. The first buckle 64 is preferably connected by a loop of webbing 72 to the elastic member 60. In the illustrated embodiment, the webbing loop 72 is about two inches wide and about four inches long. The loop of webbing 72 is preferably fixed in position on an upper end portion 74 of the elastic member 60. As a result, the first buckle 64 is fixed in position on the elastic loop 62. The first buckle 64 and the first tongue assembly 26 (on the shoulder portion 12) together form a first releasable connector 70 of the leg sling 10.

The intermediate portion 14 of the leg sling 10 preferably includes a second tongue assembly 76. The second tongue assembly 76 is preferably connected by a loop of webbing 78 to the elastic member 60. In the illustrated embodiment, the webbing loop 78 is about one inch wide and about two inches long. The loop of webbing 78 is preferably movable in position along a lower end portion 80 of the elastic member 60. As a result, the second tongue assembly 76 is movable along the lower end portion 80 of the elastic member 60.

It should be understood that the second tongue assembly 76 could be supported on the webbing loop 62 in a different manner. For example, the tongue assembly 76, which is a molded plastic piece, could be formed as one piece with a portion that extends around the webbing loop 62, with the webbing loop sliding through this plastic piece. Alternatively, if the elastic member 60 is a flat single shock cord, the second tongue assembly 78 could be sewn directly on the end of the shock cord.

The leg assembly or leg portion 16 of the leg sling 10 preferably includes a shin pad 82. The shin pad 82 is relatively wide, for example, two to three inches, and thus is adapted to spread load over an area larger than that of a corresponding strap and preferably over a relatively large area of the patient's leg and thereby provide comfort to the shin or ankle area. The shin pad 82 in the illustrated embodiment is similar in construction to the shoulder pad 20. The shin pad 82 could have another form or type of construction.

A first end portion 84 of the shin pad 82 preferably includes a webbing loop 86 that supports a second buckle 88. The second buckle 88 and the second tongue assembly 76 (on the intermediate portion 14) together form a second releasable connector 90 of the leg sling 10. The second buckle 88 is engageable with the second tongue assembly 76 in a manner as described below to connect the leg portion 16 of the leg sling 10 to the intermediate portion 14 of the leg sling.

The webbing loop 86 also preferably supports a third tongue assembly 92. The third tongue assembly 92 is engageable with a third buckle 94. The third buckle 94 and the third tongue assembly 92 together form a third releasable connector 100 of the leg sling 10. The third buckle 94 is preferably formed as one piece with a shin pad adjuster 102 supported on an adjustable length webbing strap 104 that is preferably located on a second end portion 106 of the shin pad 82. The shin pad adjuster 102 enables adjustment of the length of the shin pad 82.

The leg assembly or leg portion 16 of the leg sling 10 also preferably includes a foot strap 110. The foot strap 110 is adapted to extend around a foot shoe, a cast, or a regular shoe of the patient.

The foot strap 110 preferably includes a first strap section 112 that extends from the shin pad 82 adjacent the second end portion 106 of the shin pad. The first strap section 112 preferably extends through a foot strap adjuster 116 that is preferably adjustable to vary the length of the first strap section and, thereby, of the foot strap 110. The foot strap 110 preferably has a tie down 118 to prevent the first strap section 112 from hanging loosely. The foot strap adjuster 116 is preferably formed as one piece with a fourth buckle 124 of the leg sling 10.

The foot strap 110 preferably includes a second strap section 122 that extends from the shin pad 82 adjacent the first end portion 84 of the shin pad. The second strap section 122 preferably terminates in a fourth tongue assembly 120. The fourth buckle 124 and the fourth tongue assembly 120 together form a fourth releasable connector 130 of the leg sling. When the fourth tongue assembly 120 is engaged with the fourth buckle 124, the foot strap 110 is completed. The second strap section 122 is preferably constrained by a tie down 132.

It should be understood that the leg portion 16 of the leg sling 10 could be configured in a different manner. For example, the shin pad 82 could be formed with or replaced by a webbing strap of a material similar to the material of strap 22 to wrap around the ankle area of the patient. This would allow the leg to be carried in front of the body with the elastic member 60 positioned on the inside of the leg, rather than outside of the leg as illustrated.

To use the leg sling 10, the shoulder portion 12 of the leg sling is placed around the shoulder of the patient that is opposite the leg to be supported. This can be done, if desired, when the shoulder portion 12 is disconnected from the intermediate portion 14 and from the leg portion 16 of the leg sling 10. The shoulder pad 20 is preferably placed in a comfortable position overlying the shoulder of the patient, as shown for example in FIG. 1.

The length of the front strap 22 is preferably adjusted using the front strap adjuster 30, and the length of the back strap 24 is preferably adjusted using the back strap adjuster 34, to position the first tongue assembly 26. The first tongue assembly 26 is preferably placed in the desired front to back position, and also preferably placed in the desired vertical position. The vertical placement of the first tongue assembly 26 sets the overall length of the leg sling 10, including the height of the leg portion 16 off the ground when supported by the elastic intermediate portion 14. When the shoulder portion 12 of the leg sling 10 is thus positioned on the patient, the first tongue assembly 26 preferably faces generally downward.

The leg portion 16 of the leg sling 10 is connected with, or attached to, the leg and foot of the patient. This can be done, if desired, when the leg portion 16 is disconnected from the intermediate portion 14 and the shoulder portion 12 of the leg sling 10. The shin pad 82 is preferably wrapped around the lower leg or ankle of the patient. The length of the shin pad 82 is preferably adjusted, using the shin pad adjuster 102, so that the shin pad fits snugly around the lower leg or ankle of the patient.

The foot strap 110 is extended around the foot or shoe of the patient. The length of the foot strap 110 is preferably adjusted, using the foot strap adjuster 116, so that the foot strap fits snugly around the foot, shoe or cast of the patient. When the leg portion 16 of the leg sling 10 is thus positioned on the patient, the second buckle 88 preferably faces generally upward.

The intermediate portion 14 of the leg sling 10 is then connected with, or attached to, the leg portion 16 of the leg sling and the shoulder portion 12 of the leg sling. Specifically, the second tongue assembly 76, located on the lower end portion 80 of the elastic member 60, is engaged or connected with the second buckle 88, located on the leg portion 16 of the leg sling 10. The engagement of the second tongue assembly 76 with the second buckle 88 releasably connects the leg portion 16 of the leg sling 10 with the elastic intermediate portion 14 of the leg sling. The second releasable connector 90, which includes the second tongue assembly 76 and the second buckle, is movable along the length of the elastic member 60.

In addition, the first tongue assembly 26, located on the shoulder portion 12 of the leg sling 10, is engaged or connected with the first buckle 64, located on the upper end portion 74 of the elastic member 60. The engagement of the first tongue assembly 26 with the first buckle 74 releasably connects the shoulder portion 12 of the leg sling 10 with the elastic intermediate portion 14 of the leg sling.

When the leg sling 10 is thus completely assembled and in use on the patient, no portion of the leg sling, specifically no weight-transferring portion of the leg sling, extends around the patient's waist, and none of the weight of the supported leg is borne by the waist. The leg sling 10 extends directly between the patient's leg and the shoulder. The leg sling 10 transfers weight of from the patient's leg to the patient's opposite shoulder through the intermediate portion 14 including the elastic member 60.

The overall length of the leg sling 10 is preferably adjustable to keep the toes of the injured foot several inches off the ground, thereby providing clearance for door thresholds, curbs, etc. This is preferably done by setting the vertical position of the first releasable connector 70, using the front strap adjuster 24 and the back strap adjuster 34 on the shoulder portion 12 of the leg sling 10.

When the length of the leg sling 10 is set properly, the patient's foot is preferably held off the ground by an amount sufficient to prevent it from contact with the ground during normal walking by the patient. In addition, the foot is preferably high enough so that it does not bump on curbs, door thresholds, etc.

The elasticity of the intermediate portion 14 of the leg sling 10 provides a comfortable walking movement or standing position for the patient while using crutches or a walker. At the same time, the elasticity of the intermediate portion 14 allows the patient temporarily to extend the injured leg (as shown in dot-dash lines in FIG. 2) and place the foot on the ground, if desired, by elongating the elastic member 60 using the patient's leg muscles. This can help to prevent falls, for example.

When it is desired to remove the leg sling 10, the first releasable connector 70 is preferably released by disengaging the first tongue assembly 26 from the first buckle 64. This enables separation of the shoulder portion 12 of the leg sling from the intermediate portion 14 and the leg portion 16. The second releasable connector 90 is preferably released by disengaging the second tongue assembly 76 from the second buckle 90. This enables separation of the leg portion 16 of the leg sling 10 from the intermediate portion 14.

The shin pad is preferably released by disengaging the third tongue assembly 92 from the third buckle 94, thus opening the third releasable connector 100. The foot strap 110 is preferably released by disengaging the fourth tongue assembly 120 from the fourth buckle 124, thus opening the fourth releasable connector 130. The leg portion 16 of the leg sling can then be removed from the patient's leg, and the shoulder portion 12 can be removed from the patient's torso.

The leg portion 16 of the leg sling 10 can remain on the patient's foot. To effect this, the second releasable connector 90 can be released or opened, disengaging the intermediate portion 14 and the shoulder portion 12 of the leg sling 10 from the leg portion 16. This may be desirable while the patient sleeps, for convenience of not having to remove and reattach the leg portion 16.

It may be desirable or necessary to provide a greater range of adjustment, and a greater overall length, of the leg sling 10. Accordingly, a leg sling 10 in accordance with the present invention may include an extender assembly 140 as shown in FIG. 3.

The extender assembly 140 is preferably an elastic or non-elastic device having a fifth buckle 142 at its upper end and a fifth tongue assembly 144 at its lower end. The fifth buckle 142 is engageable with the first tongue assembly 26 on the shoulder portion 12 of the leg sling 10. The fifth tongue assembly 144 is engageable with the first buckle 64 on the elastic intermediate portion 14 of the leg sling 10. The length of the extender assembly 140 is preferably adjustable with an adjuster 146. Use of the extender assembly 140 in this manner can provide a greater range of adjustment, and a greater overall length, of the leg sling 10. The extender assembly 140 may, alternatively, be connectable between the shoulder portion 12 of the leg sling 10 and the intermediate portion 14 of the leg sling.

From the above description of the invention, those skilled in the art will perceive improvements, changes, and modifications in the invention. For example, one or more of the other structures of the leg sling could be made from an elastic material, for example, the front strap 20, the back strap 24, or the shoulder pad 20. Such improvements, changes, and modifications within the skill of the art are intended to be included within the scope of the appended claims.

Having described the invention, we claim:

1. A leg sling for supporting a patient's leg, said leg sling comprising:
a leg portion connectable with the patient's leg to transfer the weight of the patient's leg to said leg sling;
a shoulder portion extensible across a shoulder of the patient to transfer weight from said leg sling to the patient's shoulder; and
an elastic intermediate portion connected between said leg portion and said shoulder portion to transfer weight from said leg portion to said shoulder portion;
said leg sling being free of weight-transferring portions extensible around the patient's waist when in use by the patient.

2. A leg sling as set forth in claim 1 wherein said intermediate portion of said leg sling has first and second end portions, said leg sling further comprising a first connector releasably connecting said first end portion of said intermediate portion of said leg sling with said leg portion of said leg sling and a second connector releasably connecting said second end portion of said intermediate portion of said leg sling with said shoulder portion of said leg sling.

3. A leg sling as set forth in claim 2 wherein said second connector is movable between a plurality of different positions along said elastic intermediate portion of said leg sling.

4. A leg sling as set forth in claim 2 wherein said each one of said first and second connectors comprises a tongue assembly that is releasably engageable with a buckle.

5. A leg sling as set forth in claim 1 wherein said elastic intermediate portion of said leg sling comprises an elastic member having first and second end portions, a first connector part on said first end portion of said elastic member, and a second connector part on a second end portion of said elastic member.

6. A leg sling as set forth in claim 5 wherein said second connector part is movable between a plurality of different positions along said elastic member.

7. A leg sling as set forth in claim 1 wherein said elastic intermediate portion of said leg sling comprises a shock cord having a flat or circular cross-sectional configuration.

8. A leg sling as set forth in claim 1 wherein said elastic intermediate portion of said leg sling comprises a shock cord.

9. A leg sling as set forth in claim 1 wherein said elastic intermediate portion of said leg sling comprises an elastic member having a flat or circular cross-sectional configuration.

10. A leg sling as set forth in claim 1 wherein said shoulder portion of said leg sling comprises a shoulder pad disposed between first and second straps, said shoulder pad being substantially wider than said first and second straps, and wherein said leg portion of said leg sling comprises a shin pad disposed between first and second straps, said shin pad being substantially wider than said first and second straps.

11. A leg sling as set forth in claim 1 wherein said shoulder portion of said leg sling comprises a shoulder pad disposed between first and second straps each of which is independently adjustable in length, and said leg portion of said leg sling comprises a shin pad that is adjustable in length and a foot strap that is adjustable in length.

12. A leg sling as set forth in claim 11 further comprising a first connector releasably connecting said intermediate portion of said leg sling with said shoulder portion of said leg sling and a second connector releasably connecting said intermediate portion of said leg sling with said leg portion of said leg sling.

13. A leg sling as set forth in claim 1 further comprising an extender assembly connectable between said intermediate portion of said leg sling and one of said shoulder portion and said leg portion of said leg sling.

14. A leg sling as set forth in claim 1 wherein said intermediate portion of said leg sling comprises an elastic member whose amount of elongation is selected to hold the patient's leg at the desired distance off the ground temporarily while walking while allowing the leg to be placed on the ground by extending the leg with muscular action.

15. A leg sling for supporting a leg of a patient off the ground, said leg sling comprising:
a leg portion connectable with the patient's leg for movement with the patient's leg;
an elastic intermediate portion;
a shoulder portion extensible across a shoulder of the patient for transferring load from said leg sling to the shoulder of the patient; and
a first connector releasably connecting said intermediate portion of said leg sling with said shoulder portion of said leg sling;
a second connector releasably connecting said intermediate portion of said leg sling with said leg portion of said leg sling.

16. A leg sling as set forth in claim 15 wherein each one of said first and second connectors comprises a buckle and a tongue assembly.

17. A leg sling as set forth in claim 15 wherein said second connector is movable between a plurality of different positions along said intermediate portion of said leg sling.

18. A leg sling as set forth in claim 15 wherein said shoulder portion of said leg sling comprises a shoulder pad disposed between first and second straps each of which is independently adjustable in length, and said leg portion of said leg sling comprises a shin pad that is adjustable in length and a foot strap that is adjustable in length.

19. A leg sling for supporting a patient's leg, said leg sling comprising:
a leg portion connectable with the patient's leg to transfer weight from the patient's leg to said leg sling;
a shoulder portion extensible across a shoulder of the patient to transfer weight from said leg sling to the patient's shoulder; and
an elastic intermediate portion connected between said leg portion and said shoulder portion to transfer weight from said leg portion to said shoulder portion;
said intermediate portion of said leg sling comprising an elastic member whose amount of elongation is selected to hold the patient's leg at the desired distance off the ground temporarily while walking while allowing the leg to be placed on the ground by extending the leg with muscular action;
said intermediate portion of said leg sling having first and second end portions, said leg sling further comprising a first connector releasably connecting said first end portion of said intermediate portion of said leg sling with said leg portion of said leg sling and a second connector releasably connecting said second end portion of said intermediate portion of said leg sling with said shoulder portion of said leg sling, said second connector being movable between a plurality of different positions along said elastic intermediate portion of said leg sling, each one of said first and second connectors comprising a tongue assembly that is releasably engageable with a buckle;
said shoulder portion of said leg sling comprising a shoulder pad disposed between first and second straps each of which is independently adjustable in length, said shoulder pad being substantially wider than said first and second straps;
said leg portion of said leg sling comprising a shin pad disposed between first and second straps, said shin pad being substantially wider than said first and second straps, and said shin pad being adjustable in length.
